(19) United States
(12) Patent Application Publication
(10) Pub. No.: US 2014/0276479 A1
(43) Pub. Date: Sep. 18, 2014

(54) TRANSDERMAL DRUG DELIVERY SYSTEM WITH OVERLAY

(71) Applicant: Noven Pharmaceuticals, Inc., Miami, FL (US)

(72) Inventors: Viet Nguyen, Miami, FL (US); Puchun Liu, Miami, FL (US); Steven Dinh, Miami, FL (US)

(73) Assignee: Noven Pharmaceuticals, Inc., Miami, FL (US)

(21) Appl. No.: 14/208,380

(22) Filed: Mar. 13, 2014

Related U.S. Application Data
(60) Provisional application No. 61/785,247, filed on Mar. 14, 2013.

(51) Int. Cl. A61K 9/70; A61K 31/565

(52) U.S. Cl. CPC A61K 9/7069; A61K 31/565
USPC 604/290; 604/307; 427/2.31

(57) ABSTRACT

Transdermal drug delivery systems comprising a drug-containing layer, a backing layer, and an occlusive overlay layer extending beyond the perimeter of the backing layer by 3 to 6 mm in all directions are described. The systems achieve increased drug delivery over an extended time period.

### CROSS-REFERENCE TO RELATED APPLICATIONS

This application claims the benefit under 35 U.S.C. §119(e) to U.S. provisional application 61/785,247, filed Mar. 14, 2013, the contents of which are incorporated here by reference in their entirety.

### FIELD OF THE INVENTION

Described herein are transdermal drug delivery systems for the extended delivery of one or more drugs, methods of making them and methods of using them.

### BACKGROUND

Transdermal drug delivery systems for administering therapeutically effective amounts of one or more drugs (pharmaceutically active agents) are well known in the art. Transdermal devices or systems can be categorized in many different ways, but those commonly called transdermal patches incorporate the pharmaceutically active agent into a carrier, usually a polymeric and/or a pressure-sensitive adhesive formulation.

Many factors influence the design and performance of transdermal drug delivery devices, such as the identity and characteristics of the drug (s), the physical and chemical characteristics of the system's components individually and their performance/behavior relative to other components, external/environmental conditions during manufacturing and storage, the properties of the topical site of application, the desired onset and rate of drug delivery, the desired drug delivery profile, and the intended duration of delivery. Cost, appearance, size and ease of manufacturing are also important considerations.

The simplest transdermal drug delivery system design provides a drug incorporated into a pressure-sensitive adhesive carrier layer (often referred to as a “monolithic” system or a “drug-in-adhesive” system), one surface of which is affixed to a backing layer and the other surface of which is affixed to a removable liner (release liner). However, when addressing all the design and performance factors and considerations to achieve the goal, this system alone cannot always exhibit the desired performance. For example, a simple monolithic system may not exhibit extended drug delivery, such as drug delivery over greater than 3 days, or over at least 7 days.

U.S. Pat. No. 5,466,465 describes an occlusive patch for microencapsulated drug that relies on water from entrapped sweat to penetrate the coat of the microcapsules to dissolve the drug, thereby permitting its release from the patch. The patch includes an adhesive layer co-terminus with the backing layer that together create a reservoir for the drug-containing matrix.

U.S. Patent Application 2004/0053901a transdermal hormone patch that includes a drug-containing polymer adhesive layer and a backing layer. The patch may optionally include or be provided with an adhesive overlay layer to provide additional adhesion or improved appearance.

WO 2009/009649 describes a multi-layer transdermal system with volatile components that has a three-layer, porous, adhesive overlay. The overlay prevents loss of the volatile component prior to use and provides increased adherence during use.

There remains a need, therefore, for a transdermal drug delivery system that can be used with many types of drugs and can provide extended drug delivery, such as drug delivery over greater than 3 days, or over at least 7 days, or longer.

### SUMMARY

In accordance with some embodiments, there are provided transdermal drug delivery systems comprising a drug-containing polymer matrix layer, a backing layer co-terminus with the polymer matrix layer, and an occlusive overlay layer extending beyond the perimeter of the polymer matrix layer and backing layer by 3 to 6 mm in all directions. In some embodiments, the overlay layer extends beyond the perimeter of the polymer matrix layer and backing layer by about 4 mm in all directions.

In some embodiments, the overlay layer is comprised of (i) a pressure-sensitive adhesive layer and (ii) a backing layer. In some embodiments, the pressure-sensitive adhesive layer of the overlay layer comprises a silicone adhesive, an acrylic adhesive, a polyisobutylene adhesive, or mixtures of two or more thereof.

In some embodiments, the drug-containing polymer matrix layer is a pressure-sensitive adhesive layer that comprises a polymer matrix comprising a drug, such as estradiol. In some embodiments, the drug-containing layer comprises a blend of an acrylic polymer, a silicone polymer, PVP, and estradiol.

In some embodiments, the system comprises an amount of drug effective to provide drug delivery over a period of time of at least 3 days, or at least 7 days.

In some embodiments, the system achieves increased drug delivery as compared to a corresponding system that does not include the overlay layer.

Also provided are methods of enhancing transdermal drug delivery of a drug from a transdermal drug delivery system comprising a drug-containing polymer matrix layer and a backing layer, comprising providing a transdermal drug delivery system comprising a drug-containing polymer matrix layer and a backing layer co-terminus with the polymer matrix layer with an occlusive overlay layer extending beyond the perimeter of the polymer matrix and backing layer by about 3-6 mm in all directions, and administering said system to a subject in need thereof wherein the method achieves increased drug delivery as compared to drug delivery from a corresponding system that does not include the overlay layer. In some embodiments, the method achieves increased drug delivery over a period of time of at least three days, or at least seven days, as compared to drug delivery from a corresponding system that does not include the overlay layer.

Also provided are methods of manufacturing a transdermal drug delivery system for enhanced transdermal drug delivery, comprising providing a transdermal drug delivery system comprising a drug-containing polymer matrix layer and a backing layer co-terminus with the polymer matrix layer with an occlusive overlay layer that extends beyond the perimeter of the polymer matrix layer and backing layer by about 1-6 mm in all directions.

### BRIEF DESCRIPTION OF THE DRAWINGS

FIG. 1A is a schematic diagram of a cross-sectional view of a transdermal delivery system 10 as described herein prior to use, and FIG. 1B is a top view thereof illustrating an overlay 24 extending beyond the perimeter of the polymer matrix 12 in all directions.

FIG. 2 illustrates serum levels (pg/ml) of estradiol over 7 days administration of a transdermal delivery system as described herein with an overlay (▪) as compared to a transdermal delivery system without an overlay (♦).

FIG. 3 illustrates the flux of estradiol (μg/cm²/hr) over 7 days from a transdermal delivery system as described herein with an overlay (▪) as compared to a transdermal delivery system without an overlay (♦).

FIG. 4 illustrates the flux of estradiol (μg/cm²/hr) over 7 days from a transdermal delivery system as described herein with an occlusive overlay (♦) as compared to a transdermal delivery system with a non-occlusive overlay (▪).

FIGS. 5A and 5B illustrate serum levels (pg/ml) of estradiol over 7 days administration of a 24 cm² transdermal delivery system without an overlay (Treatment A-♦, FIG. 5A) or with a transdermal delivery system comprising a 17 cm² polymer matrix with a 24 cm² occlusive overlay as described herein (Treatment B-; FIG. 5B), as compared to a 29 cm² commercial 3.5-day product without an overlay (Treatment D).

### DETAILED DESCRIPTION

Described herein are transdermal drug delivery systems for the extended delivery of one or more drugs, methods of making them and methods of using them. In some embodiments, the transdermal drug delivery systems achieve drug delivery over a period of time of three days or longer. In specific embodiments, the transdermal drug delivery systems include an overlay over a drug-containing polymer matrix (e.g., a drug-in-adhesive matrix such as a pressure-sensitive adhesive overlay.

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein generally means that the described composition (e.g., transdermal drug delivery system, polymer matrix, etc.) comprises less than about 5%, less than about 3%, or less than about 1% by weight, based on the total weight of the composition at issue, of the excluded component.

As used herein “subject” denotes any animal in need of drug therapy, including humans. For example, a subject may be suffering from or at risk of developing a condition that can be treated or prevented with a drug, or may be administering a drug for health maintenance purposes.

As used herein, the phrases “therapeutically effective amount” and “therapeutic level” mean that drug dosage or plasma concentration in a subject, respectively, that provides the specific pharmacological response for which the drug is administered in a subject in need of such treatment. It is emphasized that a therapeutically effective amount or therapeutic level of a drug will not always be effective in treating the conditions/diseases described herein, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

As used herein, “active surface area” means the surface area of a drug-containing layer of the transdermal drug delivery system.

As used herein, “estrogen” includes estrogenic steroids such as estradiol (17-β-estradiol), estradiol benzoate, estradiol 17β-cypionate, estropipate, equilenin, equilin, estriol, estrone, ethinyl estradiol, conjugated estrogens, esterified estrogens, and mixtures thereof.

As used herein “progestin” includes without limitation progesterone, norethindrone, norethindrone acetate, norgestimate, norgestrel, and levonorgestrel.

As used herein, “flux” (also called “permeation rate”) is defined as the absorption of a drug through skin or mucosal tissue, and is described by Fick's first law of diffusion:

J=−D(dCm/dx)

where J is the flux in g/cm²/see, D is the diffusion coefficient of the drug through the skin or mucosa in cm²/sec and dCm/dx is the concentration gradient of the drug across the skin or mucosa.

As used herein, the term “transdermal” refers to delivery, administration or application of a drug by means of direct contact with skin or mucosa. Such delivery, administration or application is also known as dermal, percutaneous, transmucosal and buccal. As used herein, “dermal” includes skin and mucosa, which includes oral, buccal, nasal, rectal and vaginal mucosa.

As used herein, “transdermal drug delivery system” refers to a system comprising a composition that releases a drug, such as estrogen, upon application to the skin (or any other surface noted above).

As used herein, “polymer matrix” refers to a polymer composition which contains one or more drugs. In some embodiments, the matrix comprises a pressure-sensitive adhesive polymer or a bioadhesive polymer. In other embodiments, the matrix does not comprise a pressure-sensitive adhesive or bioadhesive. As used herein, a polymer is an “adhesive” if it has the properties of an adhesive per se, or if it functions as an adhesive by the addition of tackifiers, plasticizers, crosslinking agents or other additives. Thus, in some embodiments, the polymer matrix comprises a pressure-sensitive adhesive polymer or a bioadhesive polymer, with a drug such as an estrogen (e.g., estradiol) dissolved or dispersed therein. The polymer matrix also may comprise tackifiers, plasticizers, crosslinking agents or other additives described herein. U.S. Pat. No. 6,024,976 describes polymer blends that are useful in accordance with the transdermal systems described herein. The entire contents of U.S. Pat. No. 6,024,976 is incorporated herein by reference.

As used herein, the term “pressure-sensitive adhesive” refers to a viscoelastic material which adheres instantaneously to most substrates with the application of very slight pressure and remains permanently tacky. A polymer is a pressure-sensitive adhesive within the meaning of the term as used herein if it has the properties of a pressure-sensitive adhesive per se or functions as a pressure-sensitive adhesive by admixture with tackifiers, plasticizers or other additives.

In some embodiments, the transdermal drug delivery system is a substantially non-aqueous, solid form, capable of conforming to the surface with which it comes into contact, and capable of maintaining such contact so as to facilitate topical application without adverse physiological response, and without being appreciably decomposed by aqueous contact during topical application to a subject.

The term pressure-sensitive adhesive also includes mixtures of different polymers and mixtures of polymers, such as polyisobutylenes (PIB), of different molecular weights, wherein each resultant mixture is a pressure-sensitive adhesive. In the last case, the polymers of lower molecular weight in the mixture are not considered to be “tackifiers,” said term being reserved for additives which differ other than in molecular weight from the polymers to which they are added.

In some embodiments, the polymer matrix is a pressure-sensitive adhesive at room temperature and has other desirable characteristics for adhesives used in the transdermal drug delivery art. Such characteristics include good adherence to skin, ability to be peeled or otherwise removed without substantial trauma to the skin, retention of tack with aging, etc. In some embodiments, the polymer matrix has a glass transition temperature (T_{g}), measured using a differential scanning calorimeter, of between about −70° C. and 0° C.

As used herein, the term “rubber-based pressure-sensitive adhesive” refers to a viscoelastic material which has the properties of a pressure-sensitive adhesive and which contains at least one natural or synthetic elastomeric polymer.

The transdermal drug delivery systems described herein include an overlay layer that is provided in addition to conventional layers, such as a backing layer, a drug-containing layer, and a (removable) release liner layer. Referring to the specific embodiment illustrated in FIG. 1A and FIG. 1B, a transdermal drug delivery system 10 comprises a polymer drug-in-adhesive matrix 12 incorporating the drug. One surface of the polymer drug-in-adhesive matrix 12 is affixed to removable release liner 14 prior to use, which release liner is removed for application to the skin or mucosa of the user. Another surface of the polymer matrix is affixed to a backing 18 which is co-terminus with the polymer matrix 12. An overlay layer 24 with a perimeter that extends beyond the perimeter of the polymer matrix layer and backing layer is affixed to the backing 18, and, in the embodiment shown, includes an overlay adhesive layer 22.

Polymer matrix 12 can comprise any polymer or adhesive suitable for formulating a transdermal drug carrier composition, including non-toxic natural and synthetic polymers known or suitable for use in transdermal systems, including solvent-based, hot melt and grafted adhesives, which may be used alone or in combinations, mixtures or blends. Examples include acrylic-based, silicone-based, rubbers, gums, polyisobutylenes, polyvinylethers, polyurethanes, styrene block copolymers, styrene/butadiene polymers, polyether block amide copolymers, ethylene/vinyl acetate copolymers, and vinyl acetate based adhesives, and bioadhesives set forth in U.S. Pat. No. 6,562,363 which is expressly incorporated by reference in its entirety. Exemplary polymer matrix compositions are described in more detail below.

The polymer matrix may include one or more of any drug suitable for transdermal administration. In some embodiments, the drug is a hydrophobic drug. In some embodiments, the drug is a hydrophilic drug (e.g., an ionizable weak base or acid). In some embodiments, the polymer matrix includes a combination of hydrophobic and/or hydrophilic drugs. In specific embodiments, the drug is an estrogen, such as estradiol. In other embodiments, the drug is a progestin, such as progesterone. In further specific embodiments, the drug comprises an estrogen and a progestin. In specific embodiments, the drug is not methylphenidate or amphetamine. In specific embodiments the drug is not encapsulated or microencapsulated.

The one or more drugs can be present in the composition in different forms, depending on which yields the optimum delivery characteristics. Thus, the drug can be in its free base form or in the form of salts, esters, or any other pharmacologically acceptable derivatives, or as prodrugs, components of molecular complexes or as combinations of these. In specific embodiments the drug, such as a salt or free base, is directly blended with the components of the polymer matrix. In some embodiments, the drug is dissolved in the polymer matrix. In other embodiments, the drug is dispersed in the polymer matrix in crystalline form. In some embodiments, a portion of the drug is dissolved in the polymer matrix and a portion is dispersed therein in crystalline form. When the polymer matrix includes more than one drug, each may independently be present in any form discussed herein.

The backing layer 18 typically is a drug impermeable backing layer or film. (By “impermeable” to the drug is meant that no substantial amount of drug loss through the backing layer is observed.) In some embodiments (as illustrated in FIG. 1A) the backing layer is adjacent one face of the carrier composition layer. When present, the backing layer protects the polymer matrix layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. In typical embodiments, the backing layer is co-terminus with the polymer matrix layer, e.g., has essentially the same size and shape of the polymer matrix layer.

The transdermal drug delivery system may include a removable release liner 14 prior to use, typically located adjacent the opposite face of the carrier composition layer as compared to the backing layer. When present, the release liner is removed from the system prior to use to expose the carrier composition layer prior to topical application. Typical release liners are silicone coated release liners and fluorocarbon coated release liners. Materials suitable for use as release liners are well-known known in the art and include the commercially available products of Dow Corning Corporation designated Bio-Release® liner and Syl-off® 7610 and 3M's 1022 Scotch Pak.

In accordance with some embodiments, the transdermal dug delivery system comprises a drug-containing polymer matrix layer (e.g., a drug-in-adhesive polymer matrix) 12. The polymer component(s) can be selected and tailored based on the drug(s) being formulated and desired pharmacokinetic and/or physical properties, as has been described previously.

In some embodiments, the polymer matrix comprises an acrylic polymer, a silicone polymer, a rubber-based polymer such as polyisobutylene or styrene-isoprene-styrene polymers, or any combination of two or more such polymers. In some embodiments, the polymer matrix comprises a blend comprising an acrylic polymer, a silicone polymer, and a soluble PVP. In some embodiments, the polymer matrix includes non-adhesive polymers, such as ethyl cellulose, which may exhibit good solubility for the drug(s) being formulated.

Acrylic Polymers

The term “acrylic polymer” is used here as in the art interchangeably with “polyacrylate,” “polyacrylic polymer,” and “acrylic adhesive.” The acrylic-based polymers can be any of the homopolymers, copolymers, terpolymers, and the like of various acrylic acids or esters. In some embodiments, the acrylic-based polymers are adhesive polymers. In other embodiments, the acrylic-based polymers function as an adhesive by the addition of tackifiers, plasticizers, crosslinking agents or other additives.

The acrylic polymer can include copolymers, terpolymers and multipolymers. For example, the acrylic polymer can be any of the homopolymers, copolymers, terpolymers, and the like of various acrylic acids. In some embodiments, the acrylic polymer constitutes from about 2% to about 95% by weight of the polymer content of the polymer matrix, including about 3% to about 90% and about 5% to about 85%, such as 2% to 95%, 3% to 90% and 5% to 85%. In some embodiments, the amount and type of acrylic polymer is dependent on the type and amount of durg (e.g., estrogen) used.

Acrylic polymers useful in practicing the invention include polymers of one or more monomers of acrylic acids and other copolymerizable monomers. The acrylic polymers also include copolymers of alkyl acrylates and/or methacrylates and/or copolymerizable secondary monomers or monomers with functional groups. Combinations of acrylic-based polymers based on their functional groups is also contemplated. Acrylic-based polymers having functional groups include copolymers and terpolymers which contain, in addition to nonfunctional monomer units, further monomer units having free functional groups. The monomers can be monofunctional or polyfunctional. By varying the amount of each type of monomer added, the cohesive properties of the resulting acrylic polymer can be changed as is known in the art. In some embodiments, the acrylic polymer is composed of at least 50% by weight of an acrylate or alkyl acrylate monomer, from 0 to 20% of a functional monomer copolymerizable with the acrylate, and from 0 to 40% of other monomers.

Acrylate monomers which can be used include acrylic acid and methacrylic acid and alkyl acrylic or methacrylic esters such as methyl acrylate, ethyl acrylate, propyl acrylate, amyl acrylate, butyl acrylate, butyl methacrylate, hexyl acrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters.

Non-functional acrylic-based polymers can include any acrylic based polymer having no or substantially no free functional groups.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

As used herein, “functional monomers or groups,” are monomer units typically in acrylic-based polymers which have reactive chemical groups which modify the acrylic-based polymers directly or which provide sites for further reactions. Examples of functional groups include carboxyl, epoxy, hydroxyl, sulfoxyl, and amino groups. Acrylic-based polymers having functional groups contain, in addition to the nonfunctional monomer units described above, further monomer units having free functional groups. The monomers can be monofunctional or polyfunctional. These functional groups include carboxyl groups, hydroxy groups, amino groups, amido groups, epoxy groups, etc. Typical carboxyl functional monomers include acrylic acid, methacrylic acid, itaconic acid, maleic acid, and crotonic acid. Typical hydroxy functional monomers include 2-hydroxyethyl methacrylate, 2-hydroxyethyl acrylate, hydroxymethyl acryl ate, hydroxymethyl methacrylate, hydroxyethyl acrylate, hydroxyethyl methacrylate, hydroxypropyl acrylate, hydroxypropyl methacrylate, hydroxybutyl acrylate, hydroxybutyl methacrylate, hydroxyamyl acrylate, hydroxyamyl methacrylate, hydroxyhexyl acrylate, hydroxyhexyl methacrylate. As noted above, in some embodiments, the acrylic polymer does not include such functional groups.

Further details and examples of acrylic adhesives which are suitable in the practice of the invention are described in Satas, “Acrylic Adhesives,” Handbook of Pressure-Sensitive Adhesive Technology, 2nd ed., pp. 396-456 (D. Satas, ed.), Van Nostrand Reinhold, New York (1989); “Acrylic and Methacrylic Ester Polymers,” Polymer Science and Engineering, Vol. 1, 2nd ed., pp 234-268, John Wiley & Sons, (1984); U.S. Pat. No. 4,390,520; and U.S. Pat. No. 4,994,267, all of which are expressly incorporated by reference in their entireties.

Suitable acrylic polymers also include pressure-sensitive adhesives which are commercially available, such as the acrylic-based adhesives sold by Henkel North America under the DURO-TAK® trade name (such as DURO-TAK® 87-2287, -4098, -2852, -2196, -2296, -2194, -2516, -2070, -2353, -2154, -2510, -9085, -9088 and 73-9301) and those sold by Cytec Industries Inc. under the GELVA® GMS trade name (such as GELVA® GMS 2480, 788, 737, 263, 1430, 1753, 1151, 2450, 2495, 3067, 3071, 3087 and 3235). Other suitable acrylic adhesives include those sold under the trademark EUDRAGIT® by Roehm Pharma GmbH, Darmstadt, Germany. For example, hydroxy functional adhesives with a reactive functional 011 group in the polymeric chain, can be used. Non-limiting commercial examples of this type of adhesives include both GELVA® GMS 737, 788 and 1151, and DURO-TAK® 87-2287-4287, -2510 and -2516.

Silicon Polymers

The term “silicone-based” polymer is used interchangeably with the terms siloxane, polysiloxane, and silicones as used herein and as known in the art. A suitable silicone-based polymer may also be a pressure-sensitive adhesive. Thus, in some embodiments, the silicone-based polymer is an adhesive polymer. In other embodiments, the silicone-based polymer functions as an adhesive by the addition of tackifiers, plasticizers, crosslinking agents, or other additives.

Suitable polysiloxanes include silicone pressure-sensitive adhesives which are based on two major components: (i) a polymer or gum and (ii) a tackifying resin. A polysiloxane adhesive can be prepared by cross-linking a gum, typically a high molecular weight polydiorganosiloxane, with a resin, to produce a three-dimensional silicate structure, via a condensation reaction in an appropriate organic, volatile solvent, such as ethyl acetate or heptane. The ratio of resin to polymer can be adjusted in order to modify the physical properties of polysiloxane adhesives. Sobieski, et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989).

Exemplary silicone-based polymers are adhesives (e.g., capable of sticking to the site of topical application), including pressure-sensitive adhesives. Illustrative examples of silicone-based polymers having reduced silanol concentrations include silicone-based adhesives (and capped polysiloxane adhesives) such as those described in U.S. Pat. No. Re. 35,474 and U.S. Pat. No. 6,337,086, which are incorporated herein by reference in their entireties, and which are commercially available from Dow Corning Corporation (Dow Corning Corporation, Medical Products, Midland, Mich.) as BIO-PSA® 7-4100, -4200 and -4300 product series, and non-sensitizing, pressure-sensitive adhesives produced with compatible organic volatile solvents (such as ethyl acetate or heptane) and available commercially under their BIO-PSA® 7-4400 series, -4500 series and -4600 series.

Further details and examples of silicone pressure-sensitive adhesives which are useful in the polymer matrices and compositions and methods described herein are mentioned in the following U.S. Pat. Nos. 4,591,622; 4,584,355; 4,585,836; and 4,655,767, which are all expressly incorporated by reference herein in their entireties. It should also be understood that silicone fluids are also contemplated for use in the polymer matrices and methods described herein.

In some embodiments, the polysiloxane constitutes from about 9% to about 97% of the polymer content of the polymer matrix, including about 8% to about 97% and about 14% to about 94%, such as 9% to 97%, 8% to 97%, and 14% to 94%.

Soluble PVP

In some embodiments, the polymer matrix includes soluble PVP. Soluble PVP has been found to be highly effective in preventing crystallization of drugs, such as estradiol, in adhesive-type transdermal drug delivery system. Soluble PVP also may modulate the transdermal permeation rate of the drug.

The term “PVP or “polyvinylpyrrolidone” refers to a polymer, either a homopolymer or copolymer, containing N-vinylpyrrolidone as the monomeric unit. Typical PVP polymers are homopolymeric PVPs and the copolymer vinyl acetate vinylpyrrolidone. The homopolymeric PVPs are known to the pharmaceutical industry under a variety of designations including Povidone, Polyvidone, Polyvidonum, Polyvidonum soluble, and Poly(1-vinyl-2-pyrrolidone). The copolymer vinyl acetate vinylpyrrolidone is known to the pharmaceutical industry as Copolyvidon, Copolyvidone, and Copolyvidonum. The term “soluble” when used with reference to PVP means that the polymer is soluble in water and generally is not substantially cross-linked, and has a molecular weight of less than about 2,000,000. See, generally, Buhler, KOLLIDON®: POLYVINYLPRYRROLIDONE FOR THE PHARMACEUTICAL INDUSTRY, BASF Aktiengesellschaft (1992).

The amount and type of soluble PVP used may depend on the quantity and type of estrogen present, as well as the type of adhesive, but can be readily determined through routine experimentation. Typically, the PVP is present in an amount from about 1% to about 20% by weight, preferably from about 5% to about 15% by weight, based on the total weight of the polymer matrix. However, the amount of PVP can be higher than 20% for example, up to 40%, depending on the particular drug used and on the desired properties of the blend. The soluble PVP may have a molecular weight of about 2,000 to 1,100,000, including 5,000 to 100,000, and 7,000 to 54,000. In some embodiments, the soluble PVP has a molecular weight of from about 17,000 to about 90,000, such as from about 17,000 to about 60,000, including from 17,000 to 90,000 and from 17,000 to 60,000.

In some embodiments, the polymer matrix comprises a soluble PVP with a rubber-based pressure-sensitive adhesive and a polyacrylate polymer, such as a blend of an acrylic polymer, a polysiloxane and a soluble PVP. In some embodiments, the blend is chosen to affect the rate of drug delivery. More specifically, a plurality of polymers including a soluble polyvinylpyrrolidone, which may have different solubility parameters for the drug and which may be immiscible with each other, may be selected to adjust the solubility of the drug in the polymer matrix, thereby controlling the maximum concentration of the drug in the system, and modulating drug delivery through the dermis.

The amount of acrylic-based polymer and silicone-based polymer can be adjusted so as to modify the saturation concentration of the drug in the polymer matrix in order to affect the rate of delivery of the drug from the system and through the skin. In some embodiments, the acrylic-based polymer and silicone-based polymer are used in a weight ratio of from about 2:98 to about 96:4, including about 2:98 to about 90:10 and 2:98 to about 86:14, such as 2:98 to 96:4, 2:98 to 90:10 and 2:98 to 86:14.

Other Polymers

As noted above, in some embodiments the polymer matrix comprises one or more rubber-based polymers, such as one or more rubber-based pressure-sensitive adhesives, such as natural or synthetic polyisoprene, polybutylene, polyisobutylene, styrene-butadiene polymers, styrene-isoprene-styrene block copolymers (such as Kraton® D111 KT), hydrocarbon polymers, such as butyl rubber, halogen-containing polymers, such as polyacrylic-nitrile, polytetrafluoroethylene, polyvinylchloride, polyvinylidene chloride, and polychlorodiene, and other copolymers thereof. Additionally or alternatively, as discussed above, the polymer matrix may comprise a non-adhesive polymer, such as ethyl cellulose and cross-linked forms of polyvinylpyrrolidone (crospovidone) such as those sold under the trade name Kollidon® CL (available from BASF).

Drug

As noted above, the polymer matrix may include one or more of any drug suitable for transdermal administration. In some embodiments, the drug is a hydrophobic drug. In some embodiments, the drug is a hydrophilic drug (e.g., an ionizable weak base or acid). In some embodiments, the polymer matrix includes a combination of hydrophobic and/or hydrophilic drugs. In specific embodiments, the drug is an estrogen, such as estradiol. In other embodiments, the drug is a progestin, such as progesterone, norethindrone acetate or levonorgestrel. In further specific embodiments, the drug comprises an estrogen and a progestin. In specific embodiments, the drug is not methylphenidate or amphetamine. In specific embodiments the drug is not encapsulated or microencapsulated. In specific embodiments, the drug is not methylphenidate or amphetamine. In specific embodiments the drug is not encapsulated or microencapsulated.

The amount of drug to be incorporated in the polymer matrix varies depending on the particular drug, the desired therapeutic effect, and the time span for which the system is to provide therapy. For most drugs, the passage of the drugs through the skin will be the rate-limiting step in delivery. A minimum amount of drug in the system is selected based on the amount of drug which passes through the skin in the time span for which the system is to provide therapy. In some embodiments, a system for the transdermal delivery of estrogen is used over a period of about 1 day, about 3 days, about 7 days, or longer. Thus, in one embodiment, the systems comprise an amount of drug (e.g., estradiol) sufficient to deliver therapeutically effective amounts of drug over a period of from 1 day to 3 days, 7 days, or longer, including for 1 day, for 2 days, for 3 days, for 4 days, for 5 days, for 6 days, for 7 days, or for longer.

When the drug is an estrogen, the concentration by weight of the estrogen in the transdermal drug delivery system is typically about 0.1 to about 50%, including about 0.1 to about 40% and about 0.3 to about 30%, such as 0.1 to 50%, 0.1 to 40% and 0.3 to 30%, all based on the total weight of the polymer matrix. In some embodiments, the estrogen is estradiol, and is present at an amount of from about 0.1 to 10%, including from about 0.1 to about 5%, such as from 0.1 to 10% and 0.1 to 5%, all based on the total weight of the polymer matrix. When the estrogen is ehtynil estradiol, typical concentrations will be lower than for estradiol, as is known in the art. Irrespective of whether there is high-loading or low-loading of the estrogen into the transdermal drug delivery system, the pressure-sensitive adhesive composition can be formulated to maintain acceptable shear, tack, and peel adhesive properties.

In some embodiments, the transdermal drug delivery system includes a therapeutically effective amount of an estrogen such as estradiol, such as from about 0.025-0.1 mg/day, including about 0.025 mg/day, about 0.0375 mg/day, about 0.05 mg/day, about 0.075 mg/day, or about 0.1 mg/day, such as 0.025-0.1 mg/day, 0.025 mg/day, 0.0375 mg/day, 0.05 mg/day, 0.075 mg/day, and 0.1 mg/day. Thus, in some embodiments, the transdermal drug delivery system comprises an amount of an estrogen such as estradiol effective to achieve a delivery of from at least about 0.025 mg to at least about 0.1 mg of estradiol per day, or the therapeutically equivalent amount of another estrogen. In some embodiments, the system comprises an amount of an estrogen such as estradiol effective to achieve a delivery of from about 0.025 mg to about 0.1 mg of estradiol per day, including about 0.025 mg/day, about 0.0375 mg/day, about 0.05 mg/day, about 0.075 mg/day, or about 0.1 mg/day, such as 0.025-0.1 mg/day, 0.025 mg/day, 0.0375 mg/day, 0.05 mg/day, 0.075 mg/day, and 0.1 mg/day, or the therapeutically equivalent amount of another estrogen. As noted above, in some embodiments, these rates are achieved over a duration of application of at least about 1 day, including at least about 3 days and at least about 7 days, such as at least 1 day, at least 2 days, at least 3 days, at least 4 days, at least 5 days, at least 6 days, and at least 7 days.

Other Components

In one embodiment, the carrier composition layer comprises a penetration enhancer. A “penetration enhancer” is an agent known to accelerate the delivery of the drug through the skin. These agents also have been referred to as accelerants, adjuvants, and sorption promoters, and are collectively referred to herein as “enhancers.” This class of agents includes those with diverse mechanisms of action, including those which have the function of improving percutaneous absorption, for example, by changing the ability of the stratum corneum to retain moisture, softening the skin, improving the skin's permeability, acting as penetration assistants or hair-follicle openers or changing the state of the skin including the boundary layer.

Illustrative penetration enhancers include but are not limited to polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin which affect the ability of keratin to retain moisture; polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered. Other agents include oleic and linoleic acids, ascorbic acid, panthenol, butylated hydroxytoluene, tocopherol, tocopheryl acetate, tocopheryl linoleate, propyl oleate, and isopropyl palmitate.

In one embodiment, the penetration enhancer is oleyl alcohol. In another embodiment, the penetration enhancer is a glycol, such as dipropylene glycol, propylene glycol, butylene glycol or polyethylene glycol. In other embodiments, the penetration enhancer comprises a mixture of at least two penetration enhancers. For example, a penetration enhancer may comprise oleyl alcohol and one or more polyhydric alcohols, such as glycerine, dipropylene glycol, butylene glycol, propylene glycol. For instance, the penetration enhancer may include oleyl alcohol and dipropylene glycol. These embodiments may be particularly useful when the drug is estradiol.

In some embodiments, a penetration enhancer is used in an amount up to about 30% by dry weight of the polymer matrix, including up to 30% by weight, up to about 20% by weight, including 20% by weight, or up to about 10% by weight, up to 10% by weight, or up to 5% by weight, including up to 5% by weight, based on the dry weight of the carrier composition layer. In some embodiments, a penetration enhancer is used in an amount of from about 5% to about 15%, such as from 5% to 15%. In specific embodiments, the penetration enhancer comprises a mixture of oleyl alcohol and dipropylene glycol which together amount to about 14% by weight of the polymer matrix. The polymer matrix may further comprise various thickeners, fillers, and other additives or components known for use in transdermal drug delivery systems.

Overlay

As noted above, the transdermal drug delivery systems described herein include an overlay layer with a perimeter that extends beyond the perimeter of the polymer matrix layer and backing layer by about 3 to about 6 mm, such as by about 4 mm, in all directions.

In specific embodiments, the overlay is an occlusive overlay. As used herein, the term “occlusive” means that the overlay exhibits a low moisture vapor transmission rate and porosity, and is substantially impermeable to water-vapor.

The overlay may be made of any material suitable for use in a transdermal drug delivery system. Examples of film materials include polyurethanes, polyolefins, metallocene polyolefins, polyesters, polyamides, polyetheresters, and A-B block copolymers, and include those sold by Kraton Polymers US under the Kraton® brand name. Typically, the film is adhesive-coated, such as by comprising a pressure-sensitive adhesive, and this adhesive overlay exhibits good adhesion. In specific embodiments, the adhesive overlay additionally or alternatively exhibits little or no interaction with the drug(s) and/or other excipients of the polymer matrix layer, such as by exhibiting low or no solubility for the drug(s) and/or other excipients of the polymer matrix layer.

In specific embodiments, the overlay 24 comprises a pressure-sensitive adhesive layer 22. As noted above, in some embodiments, the pressure-sensitive layer exhibits little or no solubility for the drug(s) (and/or other excipients) in the drug-containing polymer matrix layer. These embodiments may offer an advantage of little or no migration of the drug(s) (and/or other excipients) from the polymer matrix layer into the overlay.

For example, silicone polymers, which generally exhibit low solubility for most drugs, can be used in the pressure-sensitive adhesive layer of the overlay. Exemplary silicone polymers include silicone adhesives, such as silicone pressure-sensitive adhesives, such as silicone polymers having reduced silanol contents (including capped polysiloxane adhesives) such as those described in U.S. Pat. No. RE 35,474 and U.S. Pat. No. 6,337,086, which are incorporated herein by reference in their entireties, and polymers which are commercially available from Dow Corning Corporation (Dow Corning Corporation, Medical Products, Midland, Mich.) in the BIO-PSA® 7-4100, -4200 and -4300 product series, and non-sensitizing, pressure-sensitive adhesives produced with compatible organic volatile solvents (such as ethyl acetate or heptane) such as those available commercially under the Dow Corning Corporation BIO-PSA® 7-4400 series, -4500 series and -4600 series.

The pressure-sensitive adhesive layer of the overlay may additionally or alternatively include an acrylic polymer, such as an acrylic adhesive, and/or a polyisobutylene polymer, such as a polyisobutlylene adhesive. As noted above, in some embodiments, the adhesive is chosen to exhibit little or no solubility for the drug(s) (and/or other excipients) in the drug-containing polymer matrix layer. Suitable acrylic polymers include those discussed above, including polymers comprised of monomers such as acrylic acid, methacrylic acid, or alkyl acrylic or methacrylic esters, such as methyl acrylate, ethyl acrylate, propyl acrylate, amyl acrylate, butyl acrylate, butyl methacrylate, hexyl acrylate, hexyl methacrylate, heptyl acrylate, octyl acrylate, nonyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, tridecyl methacrylate, glycidyl acrylate, and corresponding methacrylic esters. As set forth above, non-functional acrylic-based polymers can include any acrylic based polymer having no or substantially no free functional groups. Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.

The backing layer of the overlay provides protection for the adhesive layer and may lend the overlay its occlusive property. In some embodiments, the backing layer is a soft (e.g., pliable or flexible) film. Materials suitable for use as the backing layer of the overlay include films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers.

The transdermal drug delivery systems described herein comprising an overlay layer unexpectedly achieve sustained and/or increased drug delivery as compared to a corresponding system that does not include an overlay layer. This may be reflected in any one or more of increase serum levels of the drug at a given time or cumulatively over the time period of application, increased drug flux through the skin at a given time or cumulatively over the time of application, and/or a longer period of drug delivery. In some embodiments, the drug delivery achieved by a system as described herein may be 1.2, 1.5, or more times that of a corresponding system that does not include an overlay layer.

Manufacturing Methods

The transdermal drug delivery systems described herein be prepared by methods generally known in the art. For example, the polymer matrix material can be applied to a backing layer and release liner by methods known in the art, and formed into sizes and shapes suitable for use. An overlay layer then can be applied to the backing layer such that it extends beyond the perimeter of the backing layer by about 3 to 6 mm in all directions.

For example, after the polymer matrix is formed, it may be brought into contact with a support layer, such a releaser liner layer or backing layer, in any manner known to those of skill in the art. Such techniques include calender coating, hot melt coating, solution coating, etc.

For example, a polymer matrix can be prepared by blending the components of the polymer matrix, applying the matrix material to a support layer such as a backing layer or release liner, and removing any remaining solvents. The drug (e.g., estrogen, such as estradiol) can be added at any stage. In one embodiment, all polymer matrix components, including the drug, are blended together. In another embodiment, the polymer matrix components other than the drug are blended together, and then the drug is dissolved or dispersed therein. The order of steps, amount of ingredients, and the amount and time of agitation or mixing can be determined and optimized by the skilled practitioner. An exemplary method is as follows:

Appropriate amounts of soluble PVP, solvent(s), enhancer(s), and organic solvent(s) (for example toluene) are combined and thoroughly mixed together in a vessel.

Estradiol is then added to the mixture and agitation is carried out until the drug is uniformly mixed in.

Appropriate amounts of polysiloxane and acrylic polymer are then added to the drug mixture, and thoroughly mixed.

The formulation is then transferred to a coating operation where it is coated onto a protective release liner at a controlled specified thickness. The coated product is then passed through an oven in order to drive off all volatile processing solvents. The dried product on the release liner is then joined to the backing material and wound into rolls for storage.

Appropriate size and shape “systems” are die-cut from the roll material, provided with an overlay layer, and then pouched.

Other manufacturing methods are known in the art that are suitable for making the systems described herein.

The following specific examples are included as illustrative of the transdermal drug delivery systems described herein. These example are in no way intended to limit the scope of the invention. Other aspects of the invention will be apparent to those skilled in the art to which the invention pertains.

### EXAMPLES

### Example 1

Transdermal delivery systems containing estradiol were used in a crossover clinical study with 24 subjects in which their in-vivo drug delivery profiles (PK profiles) were evaluated. The transdermal delivery systems (20 cm²) comprised the following polymer matrix with and without an occlusive overlay layer.

| POLYMER MATRIX | % w/w in |
|---|---|
| COMPONENTS | Finished Dry Product |
| Povidone 30 | 10% |
| Estradiol USP | 2.5% |
| Oleyl Alcohol | 8% |
| Dipropylene Glycol | 6% |
| Ethocel Standard | 15% |
| 4 Premium (Ethyl Cellulose) | |
| GELVA ® 788 | 20% |
| BIO-PSA ® 7-4602 | 38.5% |
| Total | 100.0% |

Backing:

DOW™ BLF 2050 Backing Layer Film (DOW™ 2050 is a 3 mil, five layer, high barrier transdermal backing layer film (EVA/PE; EVA/PE; PVDC/PVC; EVA/PE; EVA/PE) with ethylene-vinyl acetate copolymer skins and an intermediate SARAN® (polyvinylidene chloride) layer).

Overlay:

3M™ Transpore™ Surgical Tape (transparent polyethylene coated with a proprietary acrylate adhesive with a low moisture vapor transmission rate, from 3M St. Paul, Minn.).

As shown in FIG. 2, the systems with an overlay (▪) achieved increased serum levels (pg/ml) of estradiol throughout the 7 day delivery period as compared to a transdermal delivery system without an overlay (♦).

### Example 2

An in-vitro skin flux study evaluated estradiol drug flux through the skin from transdermal delivery systems with an occlusive overlay as compared to corresponding systems without an overlay.

The transdermal delivery systems comprising the following polymer matrix and overlay were used:

| POLYMER MATRIX | % w/w in |
|---|---|
| COMPONENTS | Finished Dry Product |
| Povidone 30 | 10.2% |
| Estradiol USP | 2.5% |
| Oleyl Alcohol | 8.2% |
| Dipropylene Glycol | 4.2% |
| Ethocel Standard | 15.3% |
| 4 Premium | |
| GELVA ® 788 | 20.4% |
| BIO-PSA ® 7-4602 | 39.2% |
| Total | 100.0 |

Backing:

DOW™ BLF 2050 Backing Layer Film.

Overlay Adhesive:

BIO-PSA® 7-4602 at a target coat weight of 5 mg/cm².

Overlay:

DOW™ BLF 2050 Backing Layer Film.

As shown in FIG. 3, the systems with an occlusive overlay (▪) achieved more sustained drug flux (μg/cm²/hr) throughout the 7 day delivery period as compared to a transdermal delivery system without an overlay (♦).

### Example 3

An in-vitro study evaluated drug flux through the skin from transdermal delivery systems with an occlusive overlay layer as compared to corresponding systems with a non-occlusive overlay layer.

| POLYMER MATRIX | % w/w in |
|---|---|
| COMPONENTS | Finished Dry Product |
| Povidone 30 | 10% |
| Estradiol USP | 2.5% |
| Oleyl Alcohol | 8% |
| Dipropylene Glycol | 6% |
| Ethocel Standard | 15% |
| 4 Premium | |
| GELVA ® 788 | 20% |
| BIO-PSA ® 7-4602 | 38.5% |
| Total | 100% |

Backing:

DOW™ BLF 2050 Backing Layer Film.

Overlay Adhesive: BIO-PSA® 7-4602 at a target coat weight of 5 mg/cm².

Occlusive Overlay Backing:

3M Scotchpak™ 9732 (a laminate consisting of a polyester film with ethylene vinyl acetate copolymer wherein the polyester imparts the barrier properties, from 3M St. Paul, Minn.).

Non-Occlusive Overlay Backing:

1.5 mils polyurethane film

As shown in FIG. 4, the systems with an occlusive overlay (♦) achieved greater drug flux (μg/cm²/hr) over the 7 day test period as compared to the system with a non-occlusive overlay (▪).

### Example 4

Another clinical study evaluated serum levels of estradiol during administration of transdermal delivery systems with and without use of an occlusive overlay.

For Treatment A, the transdermal drug delivery system included a 7-day drug-in-adhesive polymer matrix (24 cm²) and no overlay. Results are shown in FIG. 5. A.

For Treatment B, the transdermal drug delivery system included a 7-day drug-in-adhesive polymer matrix (17 cm²) and an occlusive overlay (24 cm²). Results are shown in FIG. 5. B.

For comparison Treatment D, two 3.5 day Vivelle® estradiol patches (29 cm²) were applied consecutively for a total application period of 7 days.

| POLYMER MATRIX | % w/w in |
|---|---|
| COMPONENTS | Finished Dry Product |
| Povidone 30 | 6.5% |
| Estradiol USP | 1.6% |
| Oleyl Alcohol | 7.5% |
| Ethocel Standard | 11.4% |
| 4 Premium | |
| GELVA ® 788 | 13% |
| BIO-PSA ® 7-4602 | 60% |
| Total | 100% |

Backing:

DOW™ BLF 2050 Backing Layer Film.

Overlay Adhesive:

BIO-PSA® 7-4602 at a target coat weight of 5 mg/cm².

Overlay:

DOW™ BLF 2050 Backing Layer Film.

As shown in FIG. 5, the system with an occlusive overlay (Treatment B, (▪)) achieved a more constant, sustained plasma levels of estradiol (pg/ml) over the 7 day test period as compared to the system without an overlay (Treatment A, (♦)), and as compared to the Vivelle® commercial product (Treatment D, (). Further, the system with an occlusive overlay (Treatment B, (▪)) achieved comparable plasma levels of estradiol even though it comprised a smaller drug-in-adhesive polymer matrix (17 cm² as compared to 24 cm² or 29 cm²).

These examples demonstrate that transdermal drug delivery systems as described herein having an occlusive overlay layer that extends beyond the perimeter of the backing layer by about 3 to 6 cm in all directions unexpectedly achieve increased drug delivery as compared to a corresponding system without an overlay, and as compared to a corresponding system with a non-occlusive overlay.

What is claimed is:

1. A transdermal drug delivery system comprising a drug-containing polymer matrix layer, a backing layer co-terminus with the polymer matrix layer, and an occlusive overlay layer extending beyond the perimeter of the polymer matrix layer and backing layer by 3 to 6 mm in all directions.

2. The transdermal drug delivery system of claim 1, wherein the overlay layer extends beyond the perimeter of the polymer matrix layer and backing layer by about 4 mm in all directions.

3. The transdermal drug delivery system of claim 1, wherein the overlay layer is comprised of (i) a pressure-sensitive adhesive layer and (ii) a backing layer.

4. The transdermal drug delivery system of claim 3, wherein the pressure-sensitive adhesive layer of the overlay layer comprises a silicone adhesive, an acrylic adhesive, a polyisobutylene adhesive, or mixtures of two or more thereof.

5. The transdermal drug delivery system of claim 1, wherein the drug-containing polymer matrix layer is a pressure-sensitive adhesive layer that comprises a polymer matrix comprising a drug.

6. The transdermal drug delivery system of claim 5, wherein the drug comprises estradiol.

7. The transdermal drug delivery system of claim 6, wherein the drug-containing layer comprises a blend of an acrylic polymer, a silicone polymer, PVP and estradiol.

8. The transdermal drug delivery system of claim 1, wherein the system comprises an amount of drug effective to provide drug delivery over a period of time of at least 3 days.

9. The transdermal drug delivery system of claim 1, wherein the system comprises an amount of drug effective to provide drug delivery over a period of time of at least 7 days.

10. The transdermal drug delivery system of claim 1, wherein the system achieves increased drug delivery as compared to a corresponding system that does not include the overlay layer.

11. A method of enhancing transdermal drug delivery of a drug from a transdermal drug delivery system comprising a drug-containing polymer matrix layer and a backing layer co-terminus with the polymer matrix layer, comprising
providing a transdermal drug delivery system comprising a drug-containing polymer matrix layer and a backing layer co-terminus with the polymer matrix layer with an occlusive overlay layer extending beyond the perimeter of the polymer matrix layer and backing layer by 3 to 6 mm in all directions, and
administering said system to a subject in need thereof,
wherein the method achieves increased drug delivery as compared to drug delivery from a corresponding system that does not include the overlay layer.

12. The method of claim 11, wherein the method achieves increased drug delivery over a period of time of at least three days as compared to drug delivery from a corresponding system that does not include the overlay layer.

13. The method of claim 11, wherein the method achieves increased drug delivery over a period of time of at least seven days as compared to drug delivery from a corresponding system that does not include the overlay layer.

14. A method of manufacturing a transdermal drug delivery system for enhanced transdermal drug delivery, comprising
providing a transdermal drug delivery system comprising a drug-containing polymer matrix layer and a backing layer co-terminus with the polymer matrix layer with an occlusive overlay layer that extends beyond the perimeter of the polymer matrix layer and backing layer by 3 to 6 mm in all directions.
